(19) United States
(12) Patent Application Publication
(10) Pub. No.: US 2015/0366845 A1
(43) Pub. Date: Dec. 24, 2015

(54) PHARMACEUTICAL COMPOSITION FOR DISEASES CAUSED BY PATHOGENIC MICROORGANISMS SUCH AS ASPERGILLUS

(71) Applicants: POLA PHARMA INC., Tokyo (JP); NIHON NOHYAKU CO., LTD., Tokyo (JP)

(72) Inventors: Tsuyoshi Shimamura, Yokohama-shi (JP); Yoshiyuki Miyata, Tokyo (JP); Makoto Gotoh, Tokyo (JP)

(73) Assignees: POLA PHARMA INC.,, Tokyo (JP); NIHON NOHYAKU CO., LTD.,, Tokyo (JP)

(21) Appl. No.: 14/760,917

(22) PCT Filed: Dec. 27, 2013
(86) PCT No.: PCT/JP2013/085343

§ 371 Date: Jul. 14, 2015

(30) Foreign Application Priority Data
Jan. 28, 2013 (JP) 2013-013503
Apr. 2, 2013 (JP) 2013-076595

(51) Int. Cl. A61K 31/4178

(52) U.S. Cl. CPC A61K 31/4178

(57) ABSTRACT

An object is to provide means for precisely treating pneumonia caused by intracellular parasite, protozoa, and/or fungus. A pharmaceutical composition for pneumonia, comprising a compound represented by the following general formula (1) as an active ingredient: (In the formula, R represents a halogen atom or a hydrogen atom, and X represents a halogen atom.)

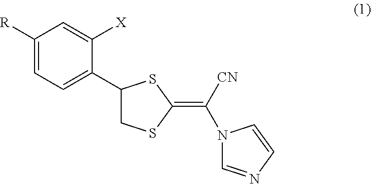

### TECHNICAL FIELD

The present invention relates to a pharmaceutical composition. In particular, the present invention relates to a pharmaceutical composition preferably usable for pneumonia.

### BACKGROUND ART

Pneumonia is caused in most cases by the secondary infection of Pneumococci as accompanied, for example, with influenza. However, fungal pneumonia, which is caused, for example, by Candida and/or Aspergillus, is rapidly increased in recent years on account of the decrease in immunity by the chemotherapy for cancer or the decrease in immunity by the onset or crisis of AIDS. On the other hand, patients infected with Chlamydia pneumonia (chlamydial pneumonia) or Trichomonas pneumonia (trichomonal pneumonia) are increased as accompanied with any particular sexual act in relation to STD in recent years. Tetracycline and macrolide antibiotic are effective on Chlamydia pneumonia, while the therapeutic agent is only metronidazole for Trichomonas pneumonia. Further, as for metronidazole, a resistant strain against metronidazole is also found in Trichomonas (see, for example, Non-Patent Document 1). In this sense, any therapeutic means, which is effective on Trichomonas pneumonia, has not been obtained in the present circumstances, although such means is demanded.

Further, in recent years, it has been confirmed that fungi such as Candida and Aspergillus co-exist in many cases in relation to Trichomoniasis (see, for example, Non-Patent Document 2 and Non-Patent Document 3). Even when Trichomonas pneumonia is cured, it is not rare that Candida pneumonia or Aspergillus pneumonia is newly caused. The following fact is considered as the cause thereof. That is, it is considered that the deterioration of the immune system is caused by the infection of Trichomonas, and the infection of any fungus is easily caused. Therefore, it is also said that a case, which occurs in an opposite manner, may arise such that any fungal disease (mycosis) is caused previously, the immune system is deteriorated, and the superinfection or coinfection with Trichomonas arises. It is said to be important that the superinfection should be avoided at the point in time of the medical treatment or therapy not only in the infectious disease caused by fungus but also in the infectious disease caused by Trichomonas. However, any therapeutic agent, which is effective on the both, does not exist until now. Further, any therapeutic agent, which is also effective on Chlamydia, does not exist as well. That is, if Trichomonas pneumonia, fungal pneumonia, and Chlamydia pneumonia are caused in a duplicated manner, any agent or drug, which can treat or cure the diseases simultaneously, is not obtained, although the agent or drug is demanded. The co-existing form as described above is extremely scarcely reported in relation to pneumonia. However, it is clinically well-known in relation to vaginitis that Trichomonas and any fungus such as Candida or the like co-exist in many cases. It is estimated that the same or equivalent situation may also exist in pneumonia. Further, the mixed infectious disease of Trichomonas and Chlamydia also exists, which is also the cause to deteriorate the prognosis after the medical treatment or therapy.

On the other hand, it is already known that pneumonia caused by fungus is cured or treated by using an antifungal agent (see, for example, Patent Document 1, Patent Document 2, Patent Document 3, and Patent Document 4). It is known that a compound such as lanoconazole or luliconazole, which is represented by the general formula (1), has the antifungal action. However, any attempt has not been made yet to cure or treat pneumonia by using lanoconazole or luliconazole. Nothing has been known at all about the fact that the compound as described above is singly used as an active ingredient and the compound as described above is used to cure or prevent pneumonia caused by any fungus such as Candida, Aspergillus or the like, pneumonia caused by protozoa such as Trichomonas or the like, pneumonia caused by intracellular parasite such as Chlamydia or the like, and pneumonia caused by a combination of any fungus, protozoa such as Trichomonas or the like, and intracellular parasite such as Chlamydia or the like.

Any compound is scarcely known, which is known as an antifungal agent and which simultaneously has an antiprotozoal action and an anticlamydial action. For example, it is known that bifonazole, miconazole, amorolfin, and butenafine, which are known as antifungal agents against athlete's foot or the like, do not have the antiprotozoal action and the anticlamydial action.

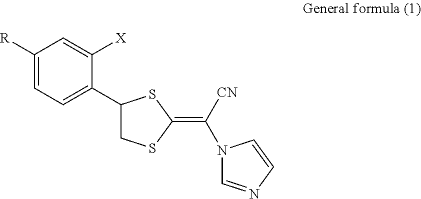

(In the formula, R represents a hydrogen atom or a halogen atom, and X represents a halogen atom.)

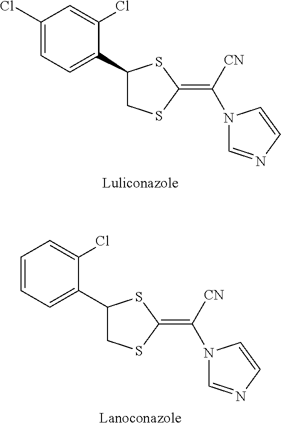 [SMILES: N#C/C(=C1/SC[C@@H](c2ccc(Cl)cc2Cl)S1)n1ccnc1 ; N#C/C(=C1/SCC(c2ccccc2Cl)S1)n1ccnc1]

### PRECEDING TECHNICAL DOCUMENTS

### Patent Documents

Patent Document 1: JP2004-521102W;
Patent Document 2: JP2002-514165W;
Patent Document 3: JP2003-527308W.

### Non-Patent Documents

Non-Patent Document 1: Pal C, Bandyopadhyay U.; “Redox-active antiparasitic drugs” Antioxid Redox Signal. 2012; 17(4):555-82;
Non-Patent Document 2: Zdrodowska-Stefanow B, Klosowska W M, Ostaszewska-Puchalska I, Bulhak-Koziol V, Kotowicz B; “Ureaplasma urealyticum and Mycoplasma hominis infection in women with urogenital diseases.” Adv Med Sci. 2006;51:250-3.
Non-Patent Document 3: Mittal A, Rastogi S, Reddy B S, Verma S, Salhan S, Gupta E; “Enhanced immunocompetent cells in chlamydial cervicitis.” J Reprod Med. 2004; 49(8):671-7

### SUMMARY OF THE INVENTION

### Technical Problem

The present invention has been made under the circumstances as described above, an object of which is to provide means for precisely treating pneumonia caused by intracellular parasite, protozoa, and/or fungus.

### Solution to Problem

Taking the foregoing circumstances into consideration, the present inventors have repeatedly performed diligent researches and efforts in order to seek for means for precisely treating pneumonia caused by intracellular parasite, protozoa, and/or fungus. As a result, it has been found out that the compound such as luliconazole and lanoconazole, which is represented by the general formula (1) described above, has the action or function to inhibit the growth of intracellular parasite such as Chlamydia or the like, protozoa such as Trichomonas or the like, and fungus such as Candida, Aspergillus or the like. It has been found out that pneumonia, which is caused by intracellular parasite, protozoa, and/or fungus, can be precisely treated by using the compound as described above as an active ingredient. Thus, the invention has been completed. That is, the present invention is as follows.

<1> A pharmaceutical composition for pneumonia, comprising a compound represented by the following general formula (1) as an active ingredient:

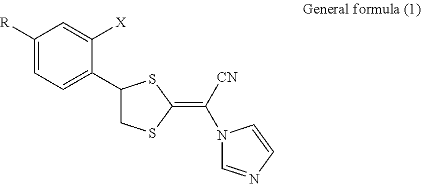

(In the formula, R represents a halogen atom or a hydrogen atom, and X represents a halogen atom.)

<2> The pharmaceutical composition for pneumonia as defined in <1>, wherein the compound represented by the general formula (1) is luliconazole or lanoconazole:

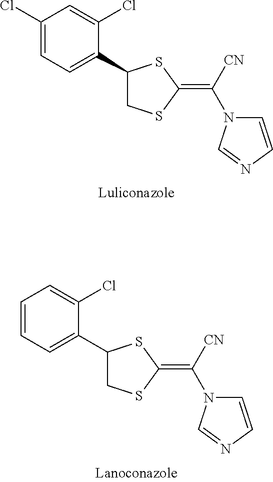

<3> The pharmaceutical composition for pneumonia as defined in <1> or <2>, wherein the pneumonia is caused by a pathogen selected from intracellular parasite, protozoa, and fungus.

<4> The pharmaceutical composition for pneumonia as defined in <3>, wherein the protozoa is protozoa belonging to genus Trichomonas.

<5> The pharmaceutical composition for pneumonia as defined in <3> or <4>, wherein the fungus is fungus belonging to genus Candida and/or fungus belonging to genus Aspergillus.

<6> The pharmaceutical composition for pneumonia as defined in any one of <3> to <5>, wherein the intracellular parasite is intracellular parasite belonging to genus Chlamydia.

<7> The pharmaceutical composition for pneumonia as defined in any one of <1> to <6>, wherein a content of the compound represented by the general formula (1) is 1 to 80% by mass with respect to a total amount of the pharmaceutical composition.

<8> The pharmaceutical composition for pneumonia as defined in any one of <1> to <7>, further containing 20 to 99% by mass of an arbitrary component for preparing a pharmaceutical preparation.

<9> The pharmaceutical composition for pneumonia as defined in any one of <1> to <8>, wherein the pharmaceutical composition is an injection agent, a tablet, an inhalation agent, or a suppository.

### Advantageous Effects of Invention

According to the present invention, it is possible to provide means for precisely treating pneumonia caused by intracellular parasite, protozoa, and/or fungus.

### BRIEF DESCRIPTION OF THE DRAWINGS

FIG. 1 shows drawings (photographs) illustrating observation results of Chlamydia inclusion bodies after the luliconazole treatment, as obtained by the fluorescence staining by using Chlamydia FA reagent “Seiken”. Panel (A) shows the observation result of Chlamydia inclusion bodies after a treatment with 8 μg/mL of luliconazole. Panel (B) shows the observation result of Chlamydia inclusion bodies after a treatment with 16 μg/mL of luliconazole. Panel (C) shows the observation result of Chlamydia inclusion bodies after a treatment with 32 μg/mL of luliconazole. In (A) and (B), dot-shaped Chlamydia inclusion bodies stained apple green were observed. No inclusion body was found in (C).

### DESCRIPTION OF EMBODIMENTS

### <1> Compound Represented by General Formula (1)

The pharmaceutical composition of the present invention is characterized in that the pharmaceutical composition contains the compound represented by the general formula (1) and the pharmaceutical composition is usable for pneumonia.

In the general formula (1), the group represented by R is a hydrogen atom or a halogen atom. The halogen atom can be preferably exemplified, for example, by chlorine atom, bromine atom, fluorine atom, and iodine atom. The group represented by R is especially preferably a hydrogen atom or a chlorine atom.

The group represented by X represents a halogen atom. The halogen atom can be preferably exemplified, for example, by chlorine atom, bromine atom, fluorine atom, and iodine atom. The group represented by X is especially preferably a chlorine atom.

The compound represented by the general formula (1) is especially preferably luliconazole (R═X═Cl; (R)-(−)-(E)-[4-(2,4-dichlorophenyl)-1,3-dithiolane-2-ylidene]-1-imidazolyl acetonitrile) and lanoconazole (R═H, X═Cl; 4-(2-chlorophenyl)-1,3-dithiolane-2-ylidene-1-imidazolyl acetonitrile), and luliconazole is especially preferred. The compound as described above suppresses the growth of intracellular parasite such as Chlamydia or the like and protozoa such as Trichomonas or the like, and the compound as described above also suppresses the growth of fungus such as Candida, Aspergillus or the like.

The compound as described above can be synthesized, for example, in accordance with a method described in JP60-218387A. That is, 1-cyanomethylimidazole and carbon disulfide are reacted to obtain a compound of (III) which is reacted with a compound of the general formula (II) having a leaving group. Thus, it is possible to obtain the compound represented by the general formula (1) as described above. The leaving group as described above can be preferably exemplified, for example, by methanesulfonyloxy group, benzenesulfonyloxy group, p-toluenesulfonyloxy group, and halogen atom.

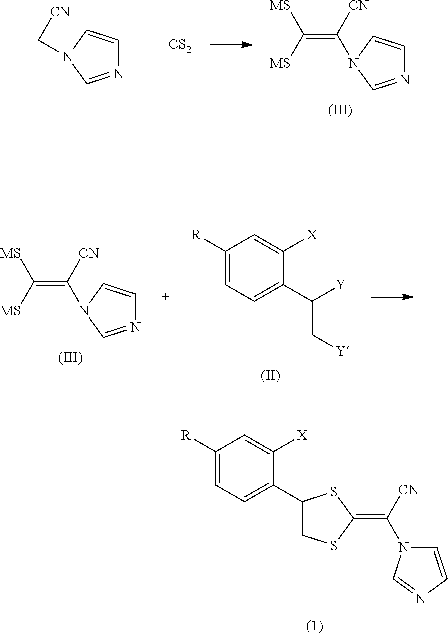

In the formula, Y, Y′ represent the leaving group such as methanesulfonyloxy group, benzenesulfonyloxy group, p-toluenesulfonyloxy group, and halogen atom, and M represents alkali metal.

In order that the compound represented by the general formula (1) exhibits the anti-intracellular parasite action, the anti-protozoa action (antiprotozoal action), and the antifungal action, it is preferable that the compound represented by the general formula (1) is contained in the pharmaceutical composition of the present invention usually by 0.5 to 80% by mass, more preferably by 1 to 80% by mass, and much more preferably by 1 to 60% by mass with respect to the total amount of the pharmaceutical composition.

### <2> Pharmaceutical Composition of the Present Invention

The pharmaceutical composition of the present invention can contain any arbitrary component for preparing a pharmaceutical preparation, other than the compound represented by the general formula (1) described above. It is preferable that the component for preparing the pharmaceutical preparation is the residual part or the balance of the compound represented by the general formula (1). The component for preparing the pharmaceutical preparation is usually 20 to 99.5% by mass, preferably 20 to 99% by mass, and more preferably 40 to 99% by mass in a total amount with respect to the total amount of the pharmaceutical composition of the present invention.

The component for preparing the pharmaceutical preparation can be preferably exemplified as follows, for example, in the case of the tablet. That is, it is possible to preferably exemplify excipients such as lactose, croscarmellose and the like; alkali agents such as sodium carbonate, sodium hydrogencarbonate and the like; acid agents such as citric acid, lactic acid, tartaric acid and the like; coating agents such as ethyl cellulose, hydroxypropyl methylcellulose, triethyl citrate and the like; binding agents such as gum arabic and the like; disintegrating agents such as starch, crystalline cellulose, hydroxypropyl cellulose and the like; sugar coating agents such as sucrose, maltitol and the like; surfactants such as POE-cured castor oil, POE sorbitan fatty acid ester and the like; plasticizers such as triethyl citrate, caprylic capric monoglyceride, diethylene glycol monoethyl ether and the like; and lubricants such as magnesium stearate, talc and the like.

The pharmaceutical composition of the present invention can also take an agent form of injection agent. As for the agent form of injection agent, it is also possible to adopt those obtained by solubilizing inclusion complexes. Alternatively, it is also possible to allow the active ingredient to be carried, for example, by liposome, niosome, fine or minute fat body (adipose particle), or self-organizing emulsion. The component, which is preferred for the agent form as described above, can be preferably exemplified, for example, by cyclodextrin which may be modified; phospholipids such as, phosphatidylcholine, phosphatidyl acid, phosphatidylinositol, phosphatidylglycerol, phosphatidylserine and the like; self-organizing agents such as acylated tripeptide and the like; polyhydric alcohols such as glycerol, propylene glycol, and 1,3-butanediol; and surfactants such as POE-cured castor oil, POE sorbitan fatty acid ester and the like. In order to adjust the osmotic pressure, it is also possible to add electrolytes such as sodium chloride.

Additionally, it is also possible to adopt an inhalation (aspiration) pharmaceutical preparation wherein the compound represented by the general formula (1) is made into fine powder which is directly aspirated into the lung.

Additionally, it is also possible to adopt a form of suppository. In the case of the suppository, it is possible to prepare the pharmaceutical preparation by using, as the component of the pharmaceutical preparation, hydrocarbons such as Vaseline, solid paraffin, microcrystalline wax, liquid paraffin and the like; esters such as olive oil, castor oil, Witepsol, carnauba wax, Japan tallow, beeswax and the like; higher alcohols such as stearyl alcohol, cetostearyl alcohol, oleyl alcohol, benzyl alcohol and the like; and surfactants such as monoglyceryl stearate, monoglyceryl oleate, sorbitan fatty acid ester and the like.

The pharmaceutical composition of the present invention can be produced on the basis of any conventional method by using the compound represented by the general formula (1) described above and arbitrary components for preparing the pharmaceutical preparation.

As for the pharmaceutical composition of the present invention, it is possible to use any one of the pharmaceutical preparation for the absorption route passing through the gastrointestinal tract and the mucous membrane and the pharmaceutical preparation for the absorption route not passing through the gastrointestinal tract and the mucous membrane. It is preferable to use the pharmaceutical preparation for the absorption route not passing through the gastrointestinal tract. The compound represented by the general formula (1) does not exhibit the strong mutagenicity unlike metronidazole. Therefore, the compound represented by the general formula (1) can be safely administered in the mode as described above.

A preferred mode of application can be appropriately selected while considering, for example, the body weight, the age, the sexuality, and the symptoms or condition of the patient. However, in the ordinary case of an adult, it is appropriate to perform the administration once or several times per several days orally or parenterally for example, by injection, suppository, or inhalation) so that the administration amount of the compound represented by the general formula (1) is 0.1 to 10 g, and such a treatment is performed for about 1 week to 3 months.

The compound represented by the general formula (1) has the anti-intracellular parasite action, the anti-protozoa action (antiprotozoal action), and the antifungal action against the intracellular parasite, the protozoa, and the fungus. The pharmaceutical composition of the present invention has been achieved on the basis of such knowledge acquired by the present inventors.

That is, the disease, to which the pharmaceutical composition of the present invention is applicable, can be the pneumonia which is caused by a pathogen selected from intracellular parasite, protozoa, and/or fungus (for example, pneumonia diagnosed that the pathogen is intracellular parasite, protozoa, and/or fungus).

In this context, the “pharmaceutical composition for pneumonia caused by the pathogen of protozoa of the present invention” can be applied to the pneumonia in which the pathogen is protozoa and the pneumonia in which the pathogen is protozoa and fungus and/or intracellular parasite. In consideration of the present circumstances in which, for example, there are many cases of the co-existence of protozoa and fungus and/or intracellular parasite or the secondary infection of protozoa, it is also preferable to apply the “pharmaceutical composition for pneumonia caused by the pathogen of protozoa of the present invention” to the pneumonia caused by the pathogen of fungus and/or intracellular parasite, in view of the suppression of any potential infection of protozoa and the prevention of any secondary infection. Further, the application to the pneumonia caused by the pathogen of fungus and/or intracellular parasite for the purpose as described above is also included in the scope of the present invention.

Further, the “pharmaceutical composition for pneumonia caused by the pathogen of fungus of the present invention” can be applied to the pneumonia in which the pathogen is fungus and the pneumonia in which the pathogen is fungus and protozoa and/or intracellular parasite. In consideration of the present circumstances in which, for example, there are many cases of the co-existence of fungus and protozoa and/or intracellular parasite or the secondary infection of fungus, it is also preferable to apply the “pharmaceutical composition for pneumonia caused by the pathogen of fungus of the present invention” to the pneumonia caused by the pathogen of protozoa and/or intracellular parasite, in view of the suppression of any potential infection of fungus and the prevention of any secondary infection. Further, the application to the pneumonia caused by the pathogen of protozoa and/or intracellular parasite for the purpose as described above is also included in the scope of the present invention.

Similarly, it is also possible to make the application to the pneumonia caused by the pathogen of intracellular parasite. That is, the “pharmaceutical composition for pneumonia caused by the pathogen of intracellular parasite of the present invention” can be applied to the pneumonia in which the pathogen is intracellular parasite and the pneumonia in which the pathogen is intracellular parasite and fungus and/or protozoa. In consideration of the present circumstances in which, for example, there are many cases of the co-existence of intracellular parasite and fungus and/or protozoa or the secondary infection of intracellular parasite, it is also preferable to apply the “pharmaceutical composition for pneumonia caused by the pathogen of intracellular parasite of the present invention” to the pneumonia caused by the pathogen of fungus and/or protozoa, in view of the suppression of any potential infection of intracellular parasite and the prevention of any secondary infection. Further, the application to the pneumonia caused by the pathogen of fungus and/or protozoa for the purpose as described above is also included in the scope of the present invention.

The “pharmaceutical composition for pneumonia caused by the pathogen of intracellular parasite, protozoa, and fungus of the present invention” can be applied not only to the pneumonia in which the pathogen is intracellular parasite, protozoa, and fungus but also to the pneumonia in which the pathogen is protozoa, the pneumonia in which the pathogen is fungus, and the pneumonia in which the pathogen is intracellular parasite, in view of the suppression of any potential infection of intracellular parasite, protozoa, or fungus and the prevention of any secondary infection. Further, the application to the pneumonia caused by the pathogen of protozoa, the pneumonia caused by the pathogen of fungus, and the pneumonia caused by the pathogen of intracellular parasite for the purpose as described above is also included in the scope of the present invention.

The fungus, which is the objective or target of the present invention, is not specifically limited, which is exemplified, for example, by fungi belonging to the genus Candida such as Candida albicans and the like and the genus Aspergillus.

The protozoa, which is the objective or target of the present invention, is not specifically limited, which is exemplified, for example, by protozoas belonging to the genus Trichomonas such as Trichomonas vaginalis and the like.

The intracellular parasite, which is the objective or target of the present invention, is not specifically limited, which is exemplified, for example, by intracellular parasites belonging to the genus Chlamydia such as Chlamydia trachomatis and the like.

### EXAMPLES

The present invention will be explained in further detail below as exemplified by Examples. However, the present invention is not limited to Examples described below.

### Example 1

The effect on Trichomonas vaginalis was investigated for luliconazole of the compound represented by the general formula (1). That is, 5×10⁶ cells of clinically isolated Trichomonas vaginalis were seeded in Trichomonas medium F (6.5 mL, contained in tube) produced by Fujiyakuhin Co., Ltd. containing Neutral Red as a marker, and the preculture was carried out for 72 hours (preculture). It was confirmed that Trichomonas grew, the acid was actively produced, and Neutral Red was changed to be yellow. After that, the preculture was added to Trichomonas medium F by every 100 μL in order to carry out the main culture, to which 0.5 mL of a test solution was added. In this situation, the number of protozoas in the solution of preculture was 1.5×10⁵ cells/mL. Three series of test solutions were prepared, in which the luliconazole concentrations were 200 μM (final concentration: 35.2 μM), 100 μM (final concentration: 17.6 μM), and 50 μM (final concentration: 8.8 μM), as luliconazole dissolved in 10% methanol saline solution. 0.5 mL of vehicle was added as a control. As for the vehicle, 10% methanol saline solution (final concentration: 0 μM) was used. Stirring was sufficiently performed after the addition, followed by culturing at 37° C. for 72 hours. After the completion of the cultivation, the color was discriminated, and the state of protozoas was observed by using an inverted microscope. Results are shown in Table 1. Accordingly, it is appreciated that luliconazole inhibits the growth of Trichomonas at 8.8 μM. In other words, it has been revealed that luliconazole is a substance which can inhibit the growth with respect to Trichomonas and which is clinically applicable, except for metronidazole. Further, it is also revealed that the minimum growth inhibitory concentration (MIC) is in the vicinity of 8.8 μM.

TABLE 1

| Final concentration | Color | Result of microscopic observation |
|---|---|---|
| 35.2 μM | red | no protozoa was observed |
| 17.6 μM | red | no protozoa was observed |
| 8.8 μM | yellow | protozoas were observed slightly |
| 0 μM | yellow | large number of protozoas were |
| | | observed |

### Example 2

The same or equivalent investigation as that of Example 1 was performed while changing luliconazole to lanoconazole. As a result, it becomes clear that lanoconazole also inhibits the growth of Trichomonas as well as luliconazole. It has been revealed that lanoconazole is a substance which can inhibit the growth with respect to Trichomonas and which is clinically applicable, except for metronidazole. Further, it is revealed that the minimum growth inhibitory concentration (MIC) is in the vicinity of 17.6 μM.

TABLE 2

| Final concentration | Color | Result of microscopic observation |
|---|---|---|
| 35.2 μM | red | no protozoa was observed |
| 17.6 μM | red | protozoas were observed slightly |
| 8.8 μM | yellow | large number of protozoas were observed |
| 0 μM | yellow | large number of protozoas were observed |

### Example 3

In Vitro Antifungal Activity on Candida albicans

The minimum growth inhibitory concentration (MIC) was measured by means of the broth microdilution method (medicament×2 dilution series) based on the use of BPMI 1640 medium (pH 7.0) buffered with 0.165 M morpholinopropanesulfonic acid. 100 μL of test microorganism yeast cells/sterilized physiological saline suspensions (1 to 5×10³ cells/mL) and 100 μL of media previously added with respective compounds and medium not added with compounds as a control were dispensed into respective wells of flat-bottom microculture plate. After performing the cultivation at 35° C. for 48 hours, the culture turbidities of the respective wells were measured at 630 nm to determine the minimum growth inhibitory concentration (MIC₈₀: μg/mL) as the minimum concentration of the compound at which the growth inhibition of 80% was exhibited with respect to the growth of the microorganism in the control culture (measured as the suspension). Results are shown in Table 3. It is appreciated that the excellent antifungal activity is exhibited in any case. Considering this fact in combination with Examples 1 and 2, it is clear that it is possible tosimultaneously inhibit the growth of protozoa such as Trichomonas and the growth of fungus such as Candida by using the compound represented by the general formula (1).

TABLE 3

| Microbial strain | Luliconazole | Lanoconazole |
|---|---|---|
| IFO0197 | 0.0625 | 0.125 |
| IFO0579 | 0.0313 | 0.0625 |
| IFO1269 | 0.0625 | 0.0625 |
| TIMM3164 | 0.5 | 0.5 |

### Example 4

Tablets for oral administration were manufactured in accordance with the following formulation. That is, Part A was granulated into granules which were made into tablets in accordance with the tablet making process by using a tablet making machine. Coating of the tablets was performed while spraying ethyl cellulose and triethyl citrate dissolved in ethanol. After the completion of the coating, the blowing was performed with warm air at 40° C. to perform the drying, and the tablets for oral administration were manufactured.

TABLE 4
(A)

| | | |
|---|---|---|
| Starch | 15 | parts by mass |
| Crystalline cellulose | 15 | parts by mass |
| Lactose | 20 | parts by mass |
| Luliconazole | 40 | parts by mass |
| Lactic acid | 0.5 | part by mass |
| Hydroxypropyl cellulose | 0.5 | part by mass |

(Coating agent)

| | | |
|---|---|---|
| Ethyl cellulose | 8 | parts by mass |
| Triethyl citrate | 1 | part by mass |

### Example 5

The following components were treated to manufacture tablets in the same manner as in Example 4.

TABLE 5
(A)

| | | |
|---|---|---|
| Starch | 15 | parts by mass |
| Crystalline cellulose | 15 | parts by mass |
| Lactose | 20 | parts by mass |
| Lanoconazole | 40 | parts by mass |
| Lactic acid | 0.5 | part by mass |
| Hydroxypropyl cellulose | 0.5 | part by mass |

(Coating agent)

| | | |
|---|---|---|
| Ethyl cellulose | 8 | parts by mass |
| Triethyl citrate | 1 | part by mass |

### Example 6

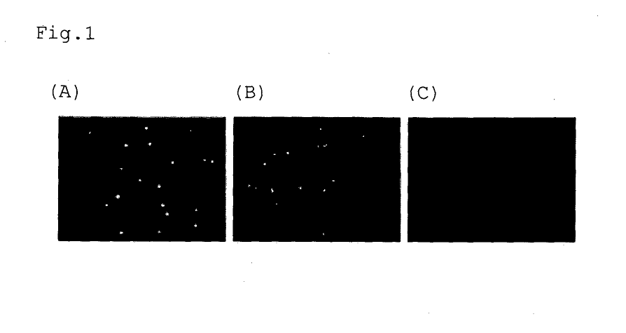

The anti-intracellular parasite action was investigated by using Chlamydia trachomatis (D/UW3/Cx). That is, Chlamydia trachomatis was cultured in the presence of ×2 dilution series of 8 to 64 μg/ml of luliconazole by using HeLa 229 cells as the host. MEM added with 8% thermally inactivated FBS, to which 1 μg/ml of cyclohexamide was added, was used as the medium, and the culture was performed for 72 hours in 5% carbon dioxide gas at 37° C. After the culture, inclusion bodies were subjected to the fluorescent staining to be apple green with Chlamydia FA reagent “Seiken” (produced by DENKA SEIKEN Co., Ltd.), and the observation was performed by using a fluorescence microscope. Results are shown in FIG. 1. Accordingly, it is appreciated that MIC of luliconazole with respect to Chlamydia trachomatis is 32 μg/ml.

### INDUSTRIAL APPLICABILITY

The present invention is applicable to pharmaceuticals.

1. A pharmaceutical composition for pneumonia, comprising a compound represented by the following general formula (1) as an active ingredient: 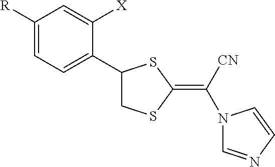
General formula (1)
wherein R represents a halogen atom or a hydrogen atom, and X represents a halogen atom.

2. The pharmaceutical composition for pneumonia according to claim 1, wherein the compound represented by the general formula (1) is luliconazole or lanoconazole:

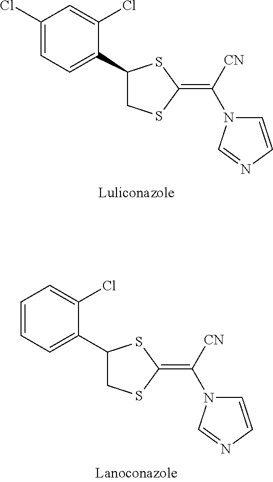

3. The pharmaceutical composition for pneumonia according to claim 1, wherein the pneumonia is caused by a pathogen selected from the group consisting of intracellular parasite, protozoa, and fungus.

4. The pharmaceutical composition for pneumonia according to claim 3, wherein the pathogen are protozoa and wherein the protozoa are protozoa belonging to genus Trichomonas.

5. The pharmaceutical composition for pneumonia according to claim 3, wherein the pathogen is fungus and wherein the fungus is fungus belonging to genus Candida and/or fungus belonging to genus Aspergillus.

6. The pharmaceutical composition for pneumonia according to claim 3, wherein the pathogen is intracellular parasite and wherein the intracellular parasite is intracellular parasite belonging to genus Chlamydia.

7. The pharmaceutical composition for pneumonia according to claim 1, wherein a content of the compound represented by the general formula (1) is to 80% by mass with respect to a total amount of the pharmaceutical composition.

8. The pharmaceutical composition for pneumonia according to claim 1, further comprising 20 to 99% by mass of an arbitrary component for preparing a pharmaceutical preparation.

9. The pharmaceutical composition for pneumonia according to claim 1, wherein the pharmaceutical composition is an injection agent, a tablet, an inhalation agent, or a suppository.

10. A method for treating pneumonia, comprising administering a pharmaceutical composition comprising a compound represented by the following general formula (1) to a subject in need thereof: 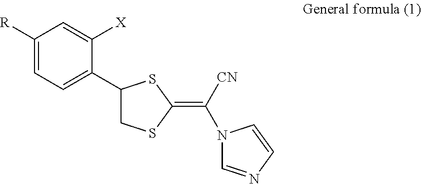
wherein, R represents a halogen atom or a hydrogen atom, and X represents a halogen atom.

11. The method according to claim 10, wherein the compound represented by the general formula (1) is luliconazole or lanoconazole:

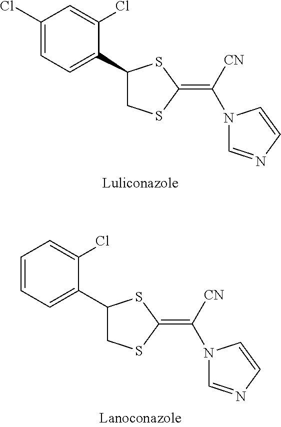

12. The method according to claim 10, wherein the pneumonia is caused by a pathogen selected from the group consisting of intracellular parasite, protozoa, and fungus.

13. The method according to claim 12, wherein the pathogen is protozoa and wherein the protozoa are protozoa belonging to genus Trichomonas.

14. The method according to claim 12, wherein the pathogen is fungus and wherein the fungus is fungus belonging to genus Candida and/or fungus belonging to genus Aspergillus.

15. The method according to claim 12, wherein the pathogen is intracellular parasite and wherein the intracellular parasite is intracellular parasite belonging to genus Chlamydia.

16. The method according to claim 10, wherein a content of the compound represented by the general formula (1) is to 80% by mass with respect to a total amount of the pharmaceutical composition.

17. The method according to claim 10, wherein the pharmaceutical composition further comprises 20 to 99% by mass of an arbitrary component for preparing a pharmaceutical preparation.

18. The method according to claim 10, wherein the pharmaceutical composition is an injection agent, a tablet, an inhalation agent, or a suppository.
